(19) United States
(12) Patent Application Publication
(10) Pub. No.: US 2017/0215858 A1
(43) Pub. Date: Aug. 3, 2017

(54) STERNOTOMY SPACING DEVICE

(71) Applicant: Rashid Mazhar Mohammad M. Haque, Doha (QA)

(72) Inventors: Rashid Mazhar Mohammad M. Haque, Doha (QA); Umair Bin Rashid Rashid Mazhar, Doha (QA)

(21) Appl. No.: 15/328,275

(22) PCT Filed: Nov. 5, 2014
(86) PCT No.: PCT/IB2014/002337

§ 371 Date: Jan. 23, 2017

(30) Foreign Application Priority Data
Jul. 23, 2014 (QA) QA/201407/00268

(51) Int. Cl. A61B 17/02

(52) U.S. Cl. CPC A61B 17/0206

(57) ABSTRACT

A device and a method for keeping the sternal edges apart for the procedure of delayed sternal closure is disclosed in which one or more, specifically shaped struts or stents are insinuated horizontally between the edges of the open sternum, keeping the two edges of the divided sternum apart by a distance determined by the length of the middle bar of the device. The disclosed device has lateral ends which are manually engaged to the medial ends of the sternal edges, by an insinuating movement while the said lateral ends of the apparatus hold onto the medial portion of the sternal edges by a passive, cupping grip which can be further secured by anchoring sutures. The whole of the disclosed device is in the form of a single piece, without any moving parts, joints, screws or nuts & bolts. The present device provides a method and an instrument which occupies minimum inter-sternal space and provides maximum sub-sternal space for accommodating cardiac oedema.

### CROSS-REFERENCE TO RELATED APPLICATIONS

This application claims priority to International Application Number PCT/IB2014/002337, filed on 5 Nov. 2014, which claims benefit of QA Patent Application Serial No. QA/201407/00268, filed 23 Jul. 2014, the disclosures of which are hereby expressly incorporated by reference in their entireties.

### FIELD OF THE INVENTION

The present invention relates to the field of Cardio-Thoracic surgery. The present invention particularly relates to device and method for keeping the Sternotomy edges apart, in a stable position, for the procedure of delayed closure of the Sternum.

### BACKGROUND OF THE INVENTION

Following prolonged cardiac operations, in a subset of patients, the heart and the mediastinal tissues swell-up to such a degree that closure of the sternum produces significant compression of the heart, resulting in circulatory compromise (cardiac temponade). In such a condition the surgeons keeps the sternum open and after a variable period of stabilization, sternum is closed; a procedure referred to as “Delayed Sternal closure” (DSC). DSC is also practiced in cases of intractable arrhythmia or coagulopathy following cardiac surgery or after placement of a circulatory assist device. DSC may be needed in >4% adult cardiac surgical. In the adult CABG plus valve surgery subgroup, from 5-12% patients may need DSC. In the pediatrics age group the collective incidence of DSC is around 7%; while amongst the infants and in the neonatal age groups, DSC is used in 22% to 61% cases, respectively.

The sternum may need to be kept open for up to several days. This time interval is required for the Myocardial & mediastinal tissue oedema to subside and to release it from the risk of temponade, while providing quick access to the chest cavity for clearance of clots & manual resuscitation. The patients remain ventilated during this period of time.

For DSC to achieve its objectives not only extra sub-sternal space is to be provided but the thoracic cage needs to be stabilized as well. In the absence of Sternal stability, the respiratory support as well as the nursing care and positioning of the patients are compromised. Furthermore, the sternal edges may injure the right ventricle when the patient is moved.

Traditionally, surgeons have been using barrels of plastic syringes or chest tubes, hand-crafted “on the table” to create such stents. At best these “on-the-spot-engineered” stents are an approximate fit for the purpose, result in unstable thoracic cage, their curved under surface encroach upon the mediastinal space, allows blood/fluid to accumulate inside the tubes increasing the risk of infection and carry potentially harmful sharp edges. Hence a reliable and strong Sternal Stenting is essential during DSC, to achieve this function, a stenting device which keeps the Sternotomy edges apart, is provided in the present invention.

Besides, the act of cutting the syringes/chest tubes into a required shape, on the operating table, is not only cumbersome but could also be injurious to the fabricating surgeon. This is something the surgeons can do away with at the end of a difficult and prolonged operation.

It is known in the state of the art, the Article: TOSHIO KANEDA ET AL: “An effective device to keep the sternum open” published in SURGERY TODAY; OFFICIAL JOURNAL OF THE JAPAN SURGICAL SOCIETY, SPRINGER-VERLAG TO, vol 29, no 2, 1 Feb. 1999 pages 194-195. In this article, it is disclosed a delayed sternal closure following cardiothoric surgery that facilitates the treatment of the heart failure and arrhythmias caused by sternal closure and also allows access to treat uncontrollable bleeding.

### SUMMARY OF THE PRIOR ART

1. Reference may be made to publish patent U.S.2009259109A1: Sternum retractor device. Besides, the same device of that patent application is described in the published article by Santini F, F. Onorati, G. Faggian, and A. Mazzucco titled “Safety and efficacy of a novel temporary sternal spreader in the management of severe post cardiotomy cardiogenic shock: A preliminary report study”, in the Journal of Thoracic & cardiovascular surgery (2011:142, 1276-1278).

The device present in the patent application and the publication is radically different in its design, principles, and utility when compared to the present invention.

The device described in the published patent application U.S.2009259109 and afore referred publication is a mechanically expandable sternal spreader. It has two diverging sternal plates with an interposing mechanism for opening/closing the spreader. The mechanism can be mechanically controlled from outside the body, by a rotating rod, to either increase or decrease the width of the open sternum. It is essentially a modified chest spreader with multi-jointed moving parts. It has two side wings and a central mechanical control box. A metallic rod comes out of the central control box & then out through the patient's body, for adjustment of the spreader's width via the threading action of the above rod which controls the spreading and closing of the spreader's side arms. The cited art has been shown in FIGS. 1 to 5.

In contrast to this published device, as is abundantly clear from the drawings of the device of the present invention (FIGS. 24-31), that it fundamentally differs from the US patent U.S.2009259109, in the following aspects:

The device of the present invention is single piece equipment. It does not have any moving parts, threading/screwing rod & cylinder or coupling mechanism and hence is free from the possibility of blood/fluids accumulating inside the device, thus significantly reducing the risks of infection to the patient. It has a specifically curved, eccentrically placed, connecting rod with an outward convexity which is flush, at its zenith, with the outer table of the Sternum. It therefore provides lowest profile with maximum sub-sternal, as well as inter-Sternal space for accommodating the oedematous (British spelling, USA writes it as Edematous) mediastinal tissues. In contrast, the bulky crock-screw mechanism producing lateral spread of the sternal plates lies within the mediastinum of the patient, thus restricting the available mediastinal space, needed for the accommodation of the oedematous myocardium & mediastinal tissues.

2: Reference may be made to another U.S. Pat. No. 6,712,821 B2 dated Mar. 30, 2004 “Sternum closure apparatus and method for helping maintain a space between parts of the sternum”

The device described in the cited art essentially comprise of two components: An elongate plate with vertical threaded shafts projecting anteriorly. This plate is approximately equal in length to the length of sternum & it occupies the space between the whole lengths of the two edges of the divided sternum, engaging to the underside of the sternum by laterally extending projections, at the level of the middle of the sternal table. The cited art has been shown in FIGS. 6 & 7; it provides a retaining/securing element, in the form of separate horizontal bar/s. After the insertion of the above elongate plate, the retaining element is fixed to the plate via its projecting vertical shafts, by means of threaded fasteners such as a nut (FIGS. 6, 7 & 8). The lateral ends of the retaining horizontal bars have underlying teeth to bite into the upper table of the sternal edge (FIGS. 9 and 10).

In contrast to this prior art, as is abundantly clear from the drawings FIGS. 24-31—that the present invention fundamentally differs from the U.S. Pat. No. 6,712,821 B2.

3: U.S. Pat. No. 6,712,821 B2, provides an “elongate plate” which restricts the mediastinal tissues at the mid-level of the Sternum. In contrast, the device of the present invention the Sternal Stent (FIGS. 24-31) has a specifically curved, eccentrically placed, connecting rod with an outward convexity which is flush, at its zenith, with the outer table of the Sternum. It therefore provides lowest profile with maximum sub-sternal, as well as inter-Sternal space for accommodating the oedematous mediastinal tissues. The “retaining mechanism of the U.S. Pat. No. 6,712,821 B2, provides “teeth” at the lateral ends of Its “retaining horizontal bars” which bite into the upper table of the sternal edge making the sternal edges vulnerable to fractures & infection. The lateral ends of the device of the present innovation are specifically spaced to firmly grip the sternal edges without any penetrating mechanism.

4: Yet another reference is made to Patent CN201179080, which is an improved version of sternal spreader for use during surgery. As depicted in FIG. 11, this is not a device for facilitating delayed sternal closure. Comparing it to the current application, it is obvious that the device described in the present invention (FIGS. 24-31) is radically different from the instrument described in the CN patent no. 201179080 in its design, principles, utility and advantages to the patient.

5: Reference is made to the U.S. Pat. No. 4,944,753. As depicted in FIGS. 12 & 13, this is an apparatus for avoiding damage to the heart during re-Sternotomy. This prior art describes a permanent sub-sternal implant, placed during the primary closure of the sternum, so that if the same patient requires mid-Sternotomy at a later time period in life, the implant would protect the heart from the possible damage from the sternal saw used during the re-Sternotomy operation.

It is obvious that the device described in the present invention (FIGS. 24-31) is radically different from the instrument described in the U.S. Pat. No. 4,944,753 in its purpose, design & utility.

6: Reference is made to the U.S. Pat. Nos. 6,569,166 B2 & 6,402,754 B1; this apparatus is proposed, as a surgical treatment, to increase the inspiratory capacity of the patients with advanced COPD. It provides a whole-sternum-length wedge which is permanently fixed in between the divided ends of the sternum, the chest is closed and the patient is discharged with the device in situ. Thus, device cited in this prior art is contrary to the present invention of the “Sternal Stent”, restricts the pre-cordial and the sub-sternal space in vertical dimension. In the situation of myocardial oedema, precluding sternal closure, the device of the above patent application cannot be used as it would further increase the temponade effect over the heart. This harmful effect would be due to the fact that the device of the patent serves to widen the bony sternal plate by providing a continuous plate which lies at a deeper level than the original bony sternum.

Furthermore, it obscures the whole of the mediastinum by excluding it from surgical view and from possibility of any urgent surgical intervention, e.g., suction, evacuation of clots etc. (FIGS. 14, 15). Hence, in contrast to this prior art, it is abundantly clear from the drawings of one of the embodiments of the device of the present innovation (FIGS. 24-31) that the present invention fundamentally differs from the U.S. Pat. Nos. 6,569,166 B2 & 6,402,754 B1 in its purpose, design and utility.

7: Reference is made to article titled “Description of a Reusable Device for the Temporary Stenting of the Open Sternum” by Kusber Hubert, M. Chares & M.-J.Polonius have published in the Journal of Thoracic & cardiovascular surgery (2002; 50: 117-119) their innovation in this article

The device described in this article has 3 components which are assembled by coupling a male and female threading mechanism. The device needs to be assembled prior to use and its breadth can be adjusted by winding or unwinding the length of the screwing/threading mechanism. It has a uniformly straight, cylindrical bar, connecting the sternal ends. The connecting bar has its superficial & deep surfaces in alignment with the outer & the inner tables of the sternum, respectively (FIGS. 16-19).

In contrast to this published device, it is abundantly clear from the attached drawings of the present innovation (FIGS. 24-31) that the present invention fundamentally differs from this published invention in the following aspects:

It is single piece equipment; it does not need to be assembled before use; it does not have any threading/screwing rod & cylinder parts or coupling mechanism and hence is free from the possibility of blood/fluids accumulating inside the rod-cylinder coupling arrangement, thus significantly reducing the risks of infection to the patient. It has a specifically curved, eccentrically placed, connecting rod with an outward convexity which is flush, at its zenith, with the outer table of the Sternum. It therefore provides lowest profile with maximum sub-sternal, as well as inter-Sternal space for accommodating the oedematous mediastinal tissues. In contrast, the horizontal connecting bar of the published article restricts the mediastinal tissues at the level of the inner table of the Sternum, besides encroaching upon the inter-Sternal space.

It has specifically placed notches and grooves over the sternal ends, allowing secure anchoring of the stent to the sternal halves, thus providing maximum stability to the thoracic cage for proper nursing care and ventilation.

Referring to the preceding discussion under published article, it becomes abundantly clear that the present innovation is radically different from the instrument described in the published article in its design, principles, utility and advantages to the patient.

8: Reference is made to article by Osaka S, Ohsawa H, Miyazawa A, Honda J. have published in the Journal of Cardiac Surgery (2000 September-October;15(5):330-332) their innovation in the article titled “Simple sternal metal stent for delayed sternal closure”. The device described in this article is a single piece; “U” shaped frame with laterally projecting side flanges at the top (FIGS. 20-21).

In contrast to this published device, as is abundantly clear from attached drawings of the present device (FIGS. 24-31) that the present invention fundamentally differs from the published innovation in the following aspects:

The lateral ends of the present invention clasps the Sternal edges while the metal frame of the published article has no such design. The middle part of the present invention has a specifically curved, eccentrically placed; thin connecting rod with an outward convexity which is flush, at its zenith, with the outer table of the Sternum. It therefore provides maximum substernal space for accommodating the oedematous mediastinal tissues. In contrast, the middle part of the connecting plate of the above published article lies at a level with the inner table of the Sternum, or even lower than that, thus restricting the mediastinal tissues to the level of the deeper surface of the sternum.

The middle part of the present invention has a thin though strong, low profile connecting bar which is eccentrically placed at the level of the superficial table of the sternum, thus, besides leaving the whole sub-sternal space unobstructed, it also makes available maximum inter-sternal space to accommodate the oedematous mediastinal tissues. In contrast, the middle part of the “metal frame” of the published article has a broad plate, at the level of the inner table of the sternum, which completely excludes the inter-Sternal space &poses a much greater, fixed restriction to the oedematous mediastinal tissues.

The lateral, Sternal-clasping design along with the specifically shaped low profile middle bar of the proposed innovation facilitates over-the device skin closure without the need to create lateral skin flaps: a procedure which increases the risk of infection and ischaemia of the pre-sternal soft tissues. In contrast, the laterally projecting broad plates of the “metal frame” of the cited published article necessitate creation of skin flaps to ensure skin closure over the “metal frame”.

Referring to the preceding discussion under published article, it becomes abundantly clear that the current application is radically different from the instrument described in the published article in its design, principles, utility and advantages to the patient.

9: Reference is made to another article by Dr. Aljafri A. Majid, from university of Malaya, Malaysia, have published in the Journal of “Annals of Thoracic surgery 1990;49:771-4”, his innovation in the article titled “Plastic Struts for Delayed Sternal Closure”

The device described in this article is a 5 cm long plastic tube with a steel wire threaded through it. The ends of the wire were inserted para-sternally on each side from the inside of the chest, brought out, and twisted together on the outside. Thus basically the arrangement is a loop of steel wire going around the sternum, with a central, sub-sternal portion which is sheathed in a piece of plastic tubing (FIG. 22).

In contrast to this published device, as is abundantly clear from the attached drawings of a particular embodiment of the present invention (FIGS. 24-31), that the present invention fundamentally differs from the published innovation in the following aspects: The lateral “U” shaped ends of the proposed innovation clasp the two Sternal edges, dissipating the force of separation of the edges as well as forces transmitted over the sternum during ventilation and care-giving, over a broad front, hence keeping the Sternum stable. In contrast the design of the published article has no lateral end. Its Para-sternal loop of wire makes an unstable, loose-tie around the sternum, producing an unstable, and flail thoracic cage.

In contrast to the inter-sternal-strut design of the proposed innovation, which provides a solid separation in-between the sternal edges, without any risk of sternal cut-through; the published article offers a Para-sternal wire loop which transmits all the forces of the ventilator& care-giving movements onto the sternal edges through the steel wire loop, thus carrying the risk of sternal instability by the wire cutting through the sternal edges.

The inter-sternal portion of the present invention is a solid strut of precise length which keeps the sternal edges apart by a fixed distance. The design in the published article has no strut between the sternal edges. Its intended function of separation of the sternal edges is determined by the looseness or the tightness of the twisting the para-sternal loop of the wire. The published article design thus offers a variable, unpredictable sternal edge separation which is determined by the degree of twisting of the wire ends by the individual operator.

The inter-sternal part of the plastic strut in the published article is in a downwardly convex form which lies below the plane of the inner table of the sternum, or even lower than that, thus restricting the mediastinal tissues to the level of the deeper surface of the sternum. It thus provides no inter-sternal space for accommodating the oedematous mediastinal tissues. In contrast, the proposed innovation has a specifically curved, eccentrically placed thin connecting rod with an outward convexity which is flush, at its zenith, with the outer table of the Sternum. It therefore provides maximum sub-sternal space for accommodating the oedematous mediastinal tissues.

In summary, the current application, in contrast to the above-mentioned published art, provides a fundamentally different, safer & more effective design and apparatus of keeping the sternal edges apart, at a fixed separation. It also provides maximum sub-sternal while clasping the sternal edges, eliminating the risk of cut-through by the looping wires.

Referring to the preceding discussion under published article, it becomes abundantly clear that the current application is radically different from the instrument described in the “published article-3” in its design, principles, utility and advantages to the patient.

10: Reference may be made to yet another published article by Apple Baum R E, Green D C, Sequeira A, McLaughlin J S. Use of a zipper in cardiac surgical operations. Ann Thorac Surg 1987; 43: 227—] this publication describes a surface zipper application to the skin edges of the wound. There is no device to keep the bone edges apart hence this citation is not relevant to our application.

11: Reference may be made to other publications such as

a): Kaneda T, Aoshima M, Ishigami N, Iemura J, Matsumoto T. An effective device to keep the sternum open. Surg Today. 1999; 29(2):194-5.
b): Ganesha krishnan K. Thyagarajan, Cobanoglu A, Temporary Stenting of the Sternum after Difficult Cardiac Operations. Ann ThoracSurg 1997; 64:1504-1505.
c): Satoh H, Sakai K, Koyama M, Matsuda H. Spool-like stent for the open sternum after cardiac operations. Ann Thorac Surg. 1997 February;63(2):572-4.
d): Jones S D, Fullerton D A, Campbell D N, Jaggers J, Brown J M, Smith R, Grover F L. Technique to stent the open sternum after cardiac operations. Ann Thorac Surg. 1994 October;58 (4):1186-7. All these cited publications describing the use of disposable syringes' barrels/plastic chest drain tubes, cutting at the operating table, into an approximate shape to act as sternal struts/stents. The matter discussed in these publications carries all those problems discussed in the ‘Background’ section of the specification

Thus, from the prior arts discussed herein the present invention radically differs from the described solutions under publications, by providing its specific, functional design, shape & dimensions, hence removing all the aforementioned concerns & dangers for the patients, as described in the statement of the invention and the section on “advantages”.

### STATEMENT OF THE INVENTION

According to the present invention, provides a device which firmly stents & keeps the Sternal edges apart. It further provides a stable “non-flail” thoracic cage to ensure proper ventilation and adequate nursing & physiotherapy care of the patient. The present invention provides a set of specifically designed struts, to suit various patient ages from neonate to big adults.

### ADVANTAGES OF THE PRESENT INVENTION

Some of the advantages and the object of the unique functional configuration & design of the present invention are listed as follows:
1: Provides maximum sub-sternal space to accommodate for cardiac &mediastinal tissue oedema.
2: Allows easy insinuation between the edges of the severed sternum while providing strong, a traumatic grip to the Sternal edges.
3: Allows easy & secure fixation to the patient by its strategically placed suture ligation grooves, providing sternal stability for ventilator & nursing care.
4: Provides an “off-the-shelf”, set of graduated sizes which allow the device to fit patients of various ages and sternal thickness.
5: Absence of movable parts or joints frees the present device from the need for adjustment to the dimensions of the stent.
6: Absence of moving parts avoids trapping of blood clots & fluids, thus decreasing the risk of infection.
7: All around smooth edges & surfaces, minimizing risk of myocardial injury.
8: Relative lack of occupation of the mediastinal space allows visibility& easy access to the mediastinum for various interventions.
9: Allows space for trans-sternal insertion of Intra-aortic Balloon Pump or other adjuncts.
10: Allows usage of device in multiple sizes in a single patient, giving maximal stability of the thoracic cage.

### BRIEF INTRODUCTION TO DRAWINGS

The invention will now be described solely by way of example and with reference to the accompanying drawings in which:

FIGS. 1-23 are the drawings of the referred prior art.

FIG. 24: shows a sketch of one embodiment of the present device wherein the device comprise a solid, metallic body, without any joints or mobile parts, comprising two lateral sternal ends opening in the opposite direction and a central, eccentrically placed, joining bar or strut.

FIG. 25: shows the profile view of one particular embodiment of the present device.

FIG. 26 shows the top view, from the superior or the external aspect of the stenting device of the present invention

FIG. 27: shows one particular embodiment of the present invention with a specific configuration of the intermediate bar/strut.

FIG. 28: shows another particular embodiment of the present invention with a specific configuration of the intermediate bar/strut.

FIG. 29: shows one particular embodiment of the present invention which is fabricated with strong though light-weight non-metallic material.

FIG. 30: shows one particular embodiment of the present invention with suture anchoring configuration along the lateral ends of the said stenting device.

FIG. 31: shows one particular embodiment of the present invention depicted as a set of graduated sizes.

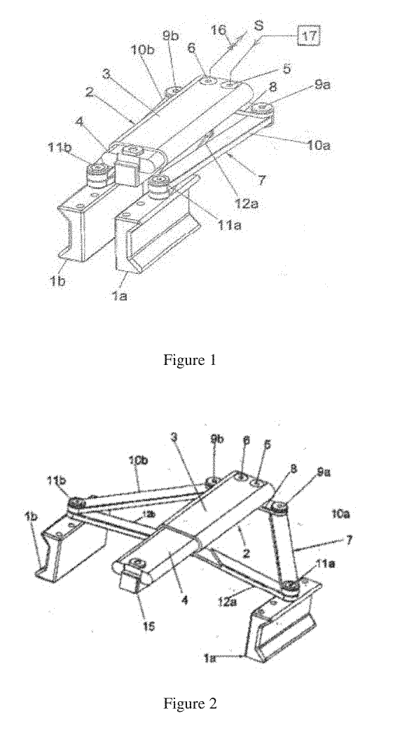

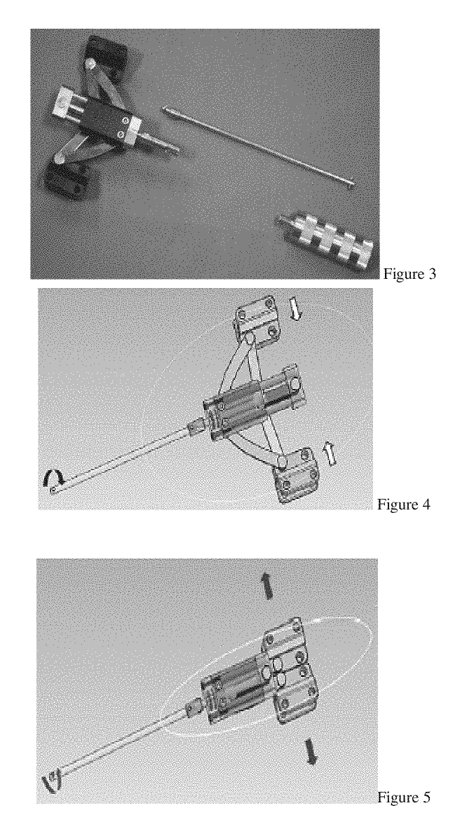

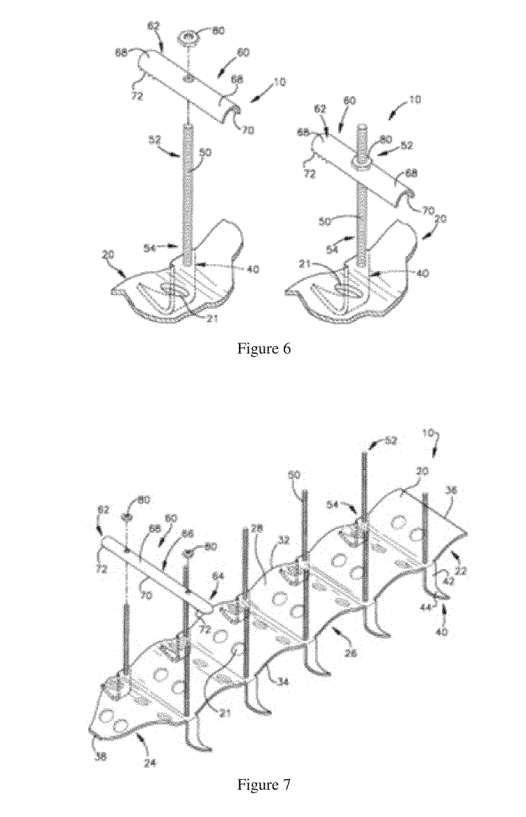

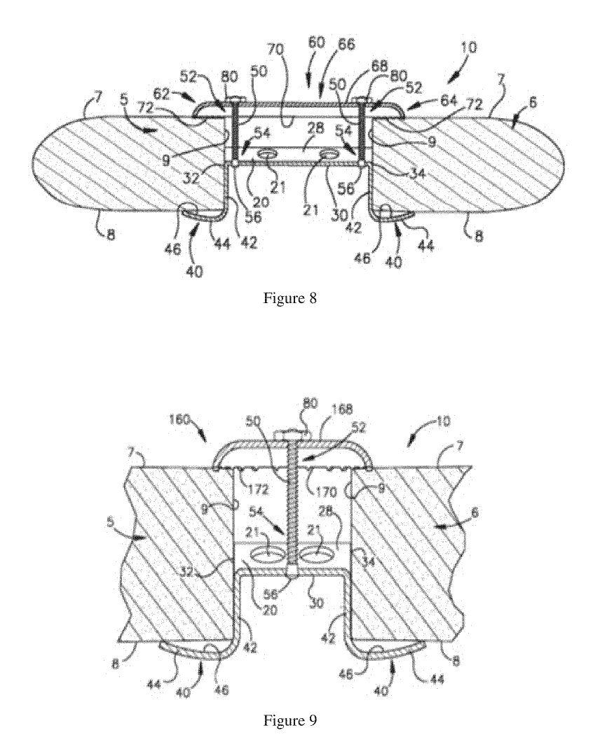

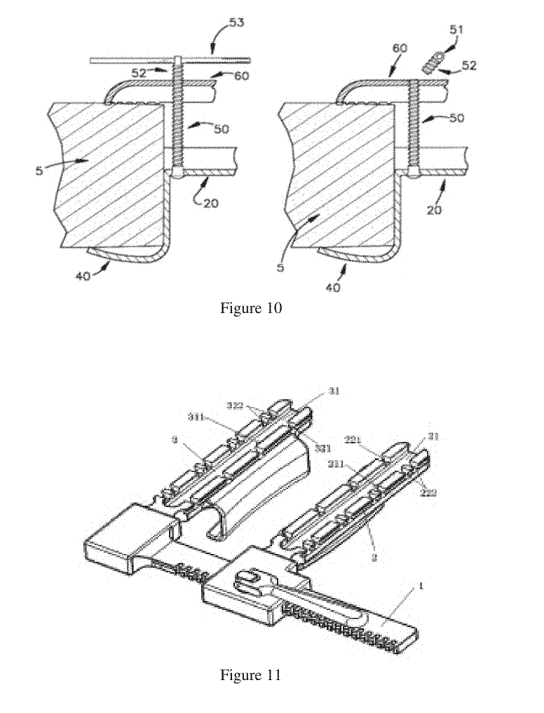

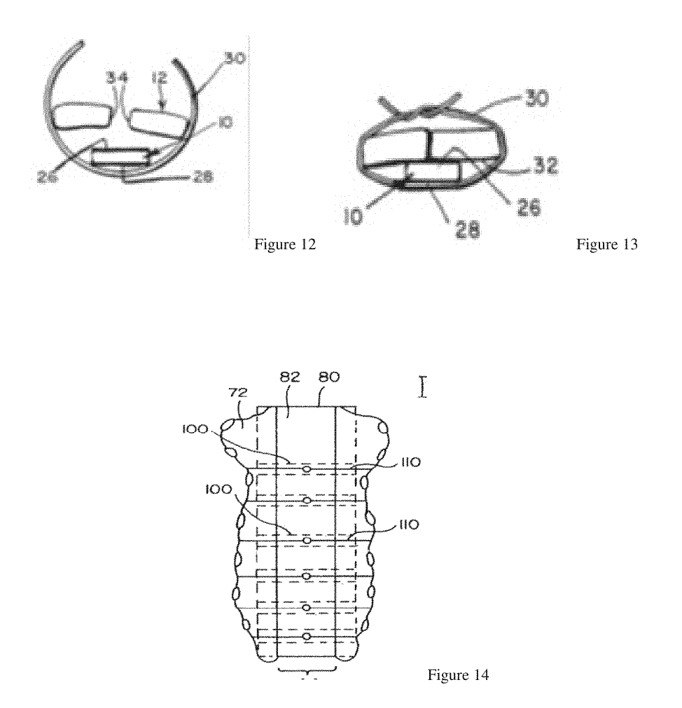

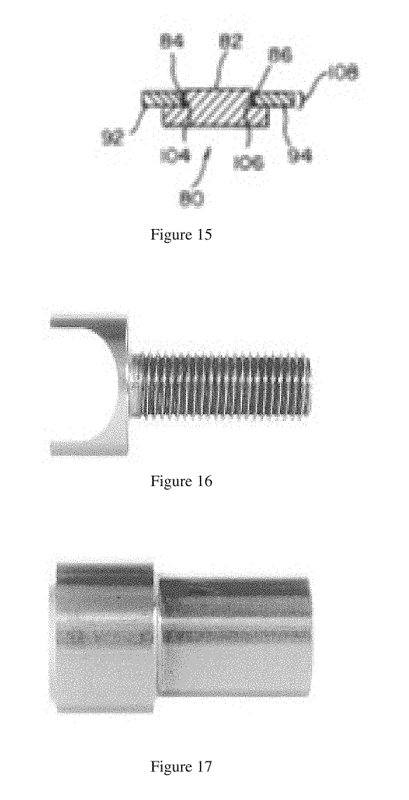

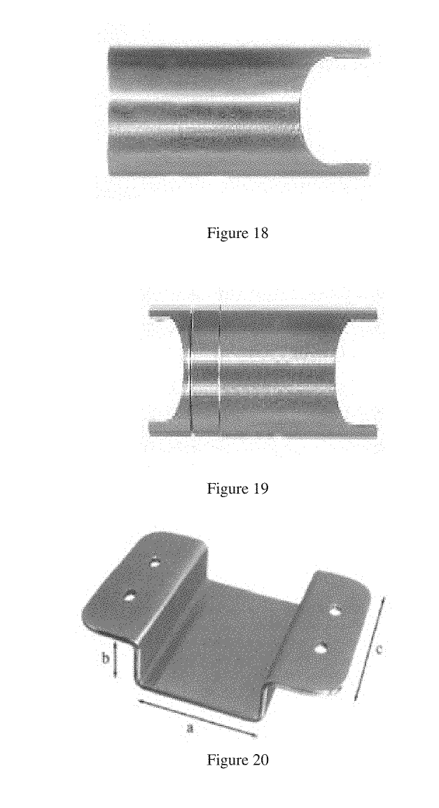

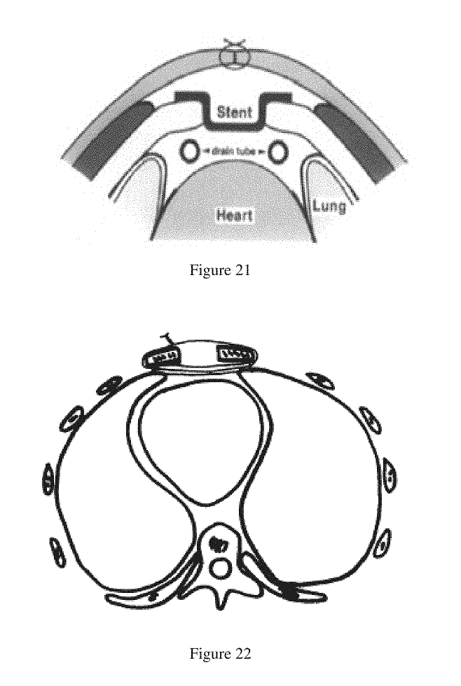

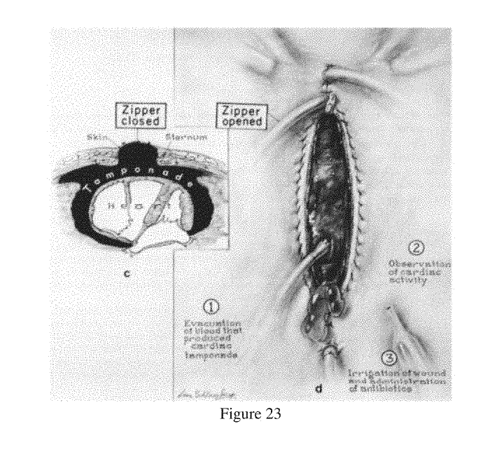

### DETAILED DESCRIPTION OF THE INVENTION

The present invention relates to the device which firmly stents & keeps the Sternal edges apart. The said device provides a fundamentally different, safer & more effective design for keeping the sternal edges apart, at a fixed separation. It also provides maximum sub-sternal while clasping the sternal edges, eliminating the risk of cut-through by the looping wires.

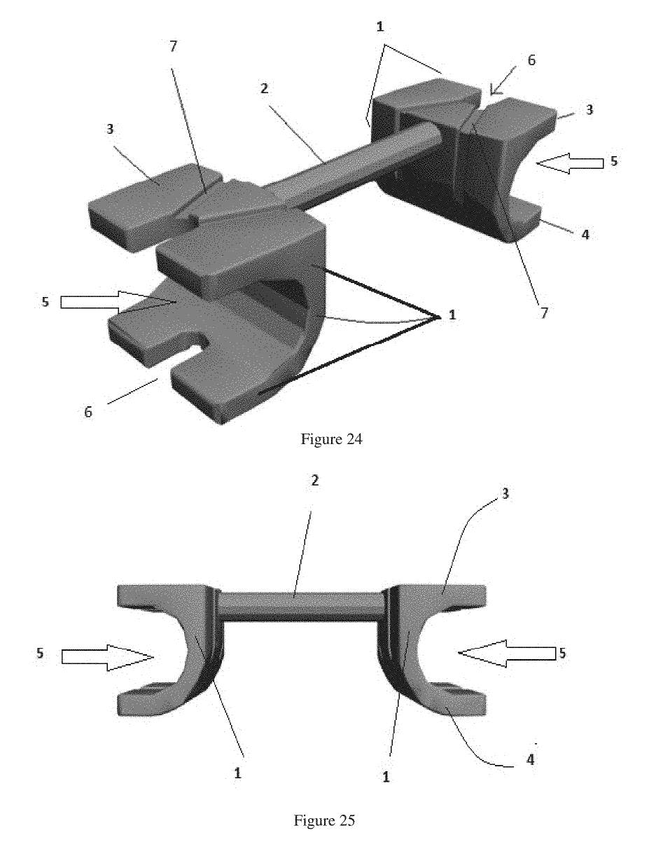

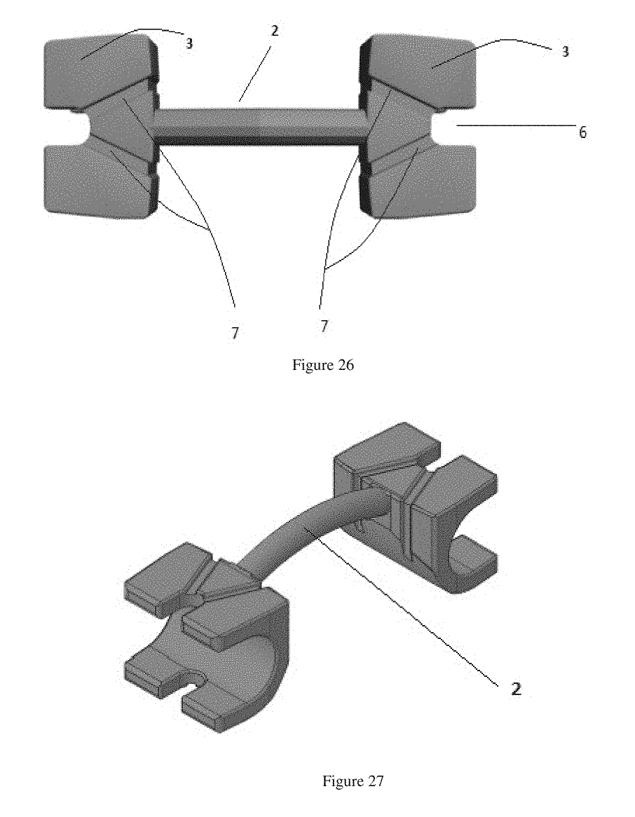

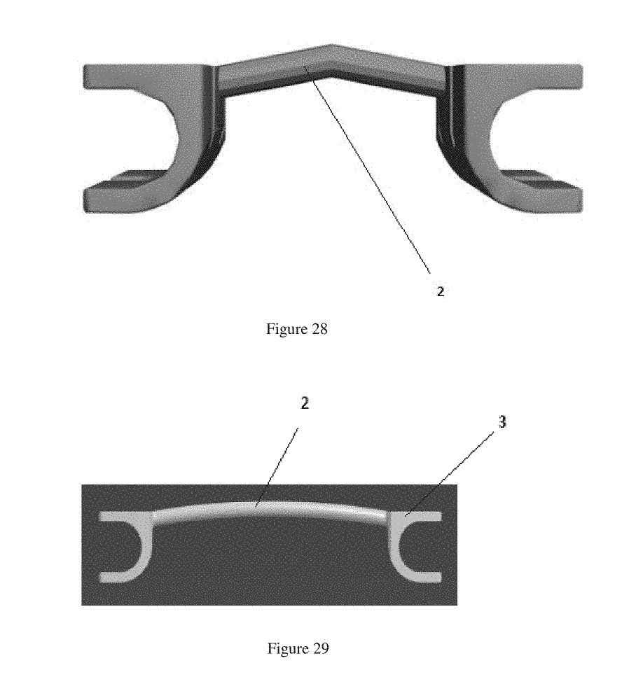

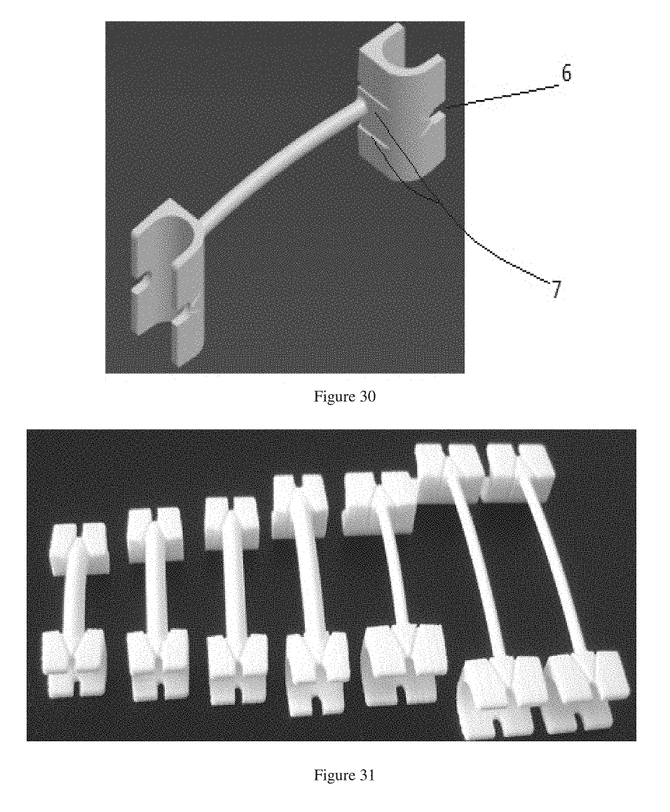

Referring to the drawings the particular embodiments of the device of the present innovation, comprises of FIG. 24, where its one particular embodiment is shown with two sternal ends (1), shaped approximately like letter “C” though opening in opposite directions. The two sternal ends (1) are interconnected by an intermediate bar/strut or elongate member (2). Each sternal end (1) has an upper lip (3) and a lower lip (4), providing a hollow cusp laterally (5) at either end. When the divided sternum is to be kept apart for delayed closure, the device of proper size is manually insinuated between the sternal edges so that its lateral end's (1), upper lip (3) lies superficial to the external table of the sternum and its lower lip (4) lies underneath the inner table of the sternal edge, thus the lateral end's hollow cusp (5) engages firmly with the divided edge of the sternum. The sternal edges are kept apart by the width and strength of the intermediate bar/strut (2). As is clear from the FIG. 24, that the interconnecting, intermediate bar (2) is placed eccentrically between the two sternal ends, thus it provides maximum sub-sternal space for the oedematous mediastinal tissues, relieving the heart from temponade effect. After engaging the sternal ends (1) to the Sternotomy edges the device could be further secured to the sternal edges by passing suture ligature through the upper & lower lips' suture ligature slot (6) and securely tying the suture by virtue of suture-holding groove (7) along the inner side of the sternal ends (1). FIG. 25 shows one sketch-in profile, of a particular embodiment of the device of the present innovation, where the eccentric, superficial position of the interconnecting bar/strut (2) is highlighted, providing maximum space in front of the mediastinal tissues to accommodate for their oedema, without compressing the heart. FIG. 25 also shows the cusp like configuration of the sternal ends (1), in this particular embodiment, whereby the upper lip (3) and the lower lip (4) of the sternal end and its hollow cusp (5) firmly engages with the medial portions of the divided sternum. FIG. 26 shows the superior aspect view of the device whereby the suture-ligature slot (6) and the two diverging suture-holding grooves (7) allow stable fixation of the device to the sternal halves by double suture ligation on both sides. FIG. 26 also highlights the width of the upper (3) and lower lips (4), in sagittal plane, providing firm anchorage over the edges of the divided sternum. FIGS. 27 & 28 depict particular embodiments of the device where the interconnecting, intermediate bar/strut (2) is either angulated or gently convex in an outward dimension, so as to provide extra space for the swollen mediastinal tissues. FIG. 29 depicts the lateral profile drawing of a device made with non-metallic, carbon-fibre reinforced Thermoplastic polymer material, exhibiting the one piece, solid fabrication with an upwardly convex interconnecting, and intermediate bar/strut (2) providing minimal occupation of mediastinal space by the device itself and at the same time providing maximum sub-sternal space for tissue oedema to expand. FIG. 30 shows the configuration of the suture-holding groove (7), extending along the medial side of the sternal end (1), providing a secure suture ligature anchoring facility. FIG. 31 shows a particular embodiment of the proposed innovation whereby a set of several devices are provided with increasing dimensions of their lateral sternal ends (1), the interconnecting bars (2) and the upper & lower lips (3 and 4) of the devices to allow the surgeon to choose one or more than one devices to be deployed in patients of varying body sizes.

Devices of varying sizes, based upon measurements of the various levels of divided sternii in different age groups (as acquired during the developmental phase of the present invention), can be deployed in the same patient, at different levels of the divided sternum, providing extra secure stenting of the sternum.

One of the major concerns with DSC is the risk of infection, particularly into the cut surfaces of the sternal bone. To help decrease this risk of infection, in one particular embodiment, the surface of the device of the present invention would be provided with a mechanism of local delivery of antibiotics. Local antibiotic delivery is known to prevent the bacterial colonization onto the device or the implant's surface, thus reducing the risk of implant-related infections. Another benefit of local delivery systems is that high concentrations of the antibiotic are achieved in the desired area without the need for high systemic doses of the antibiotics and its associated side effects.

To provide such local antibiotic delivery, the device could be either coated with antibiotics or the antimicrobial molecules could be covalently attached onto the device surfaces. Coating of the device with an antibiotic/antimicrobial agent of choice could be either without a carrier (such as spraying the device with a methanol solution containing antibiotics) or with a carrier such as sol-gel, Silicone Polymer, polylactic acid (PLA) coating, Carbonated hydroxyapatite (CHA) or poly (D, L-Lactide) (PDLLA) coating carrying specific antibiotics or various antimicrobial agents (e.g., Chlorhexidine, Polyhexamethylene Biguanides, Hydroxy apatite, Chitosan etc). Another method of antibiotic surface coating could be either by Nanopeptide coating or by using antibiotic-impregnated polymethylmethacrylate cement filling grooves made over the tissue contact areas of the device of the present invention.

In one particular embodiment of the present invention the device's surface could be permanently rendered antimicrobial by covalent attachment of antibiotics or other bactericidal peptides. The bonding does not allow the antibiotic molecules to elude off the surface of the device, thus decreasing possible local and systemic toxicity while providing long-lasting protection from infection. In such an embodiment the device would be fabricated of Titanium alloys with covalent attachment of vancomycin, Gentamycin and/or other bioactive molecules on its surfaces.

In one embodiment anti-biotic coating of the surface of the device help further decrease the risk of infection. This could be in the form of using an antibiotic impregnated metal for making the device of the present invention, example being Titanium with covalently-linked Vancomycin on its surface.

In yet another embodiment of the present invention another method of antibiotic surface coating could be by using antibiotic-impregnated polymethylmethacrylate cement filling grooves made over the tissue contact areas of the device of the proposed invention.

In another embodiment the device may be fabricated with radiolucent material of required strength; one example of such material being Carbon-fibre-reinforced Thermoplastics (e.g., Nylon, Polycarbonates & polyketones). The clinical advantage with such an embodiment of the present device would be that chest X-rays done, with the device in place, would not be obscured by the shadow of the device.

In yet another embodiment the advantages of the local antibacterial delivery and the radio-lucency attributes of the device could be combined in a single fabrication with the radio-transparent device providing local antibacterial release as well.

In yet another embodiment of the present invention, the device being provided in a set of graduated sizes to be suitable for patients of various ages and body sizes.

Thus the preferred embodiments of the present invention have been described in reference to their application environment; however these depictions are merely illustrative of the principles underlying the present invention.

### EXAMPLES

### Example 1

A working prototype of the present invention was used in 4 adult. In all adult patients the stent firmly kept the sternal edges apart and helped improve the haemodynamics of the patients. The device of the present invention as sternal stents remained in situ for an average period of 4 days. There were no instances of slippage, dislodgement or local trauma. The thoracic cage remained stable with the stents in place, allowing optimal ventilatory and nursing care of the patients. Delayed sternal closure was achieved as planned without any subsequent untoward results attributable to the use of the sternal stent devices.

### Example 2

A working prototype of the present invention was used in 12 pediatric cardiac surgical patients where chest could not be closed primarily. The pediatric patients ages ranged from 1-195 days (average age 35.6 days). In all these patients the stent firmly kept the sternal edges apart and helped improve the haemodynamics of the patients. The device of the present invention as sternal stents remained in situ for an average period of 4 days. There were no instances of slippage, dislodgement or local trauma. The thoracic cage remained stable with the stents in place, allowing optimal ventilatory and nursing care of the patients. Delayed sternal closure was achieved as planned without any subsequent untoward results attributable to the use of the sternal stent devices.

### Example 3

The device may be fabricated with radiolucent material of required strength. One example of such material is Carbon-fibre-reinforced Thermoplastics (e.g., Nylon, Polycarbonates & polyketones). Thus fabrication would do away with the obscuring of patient's X-ray picture with the projected shadow of the device in situ. An unobstructed, clear X-ray image would help improve clinical decision-making.

1. A device for keeping apart the sternal edges of a patient with mid-Sternotomy, the device comprising;
an elongate member which act as a strut between the sternal edges to keep these apart, the two outer ends of the apparatus dimensioned & configured to clasp and firmly engage with the severed edges of the sternum and provided with means of further securing the apparatus to the severed halves of the sternum; the whole device is one solid configuration, without any moving parts or joints.

2. The device as set forth in claim 1, wherein the elongate member is in the form of a horizontal bar/rod between the outer ends of the device.

3. The device as set forth in claim 1, wherein the length of the elongate member determines the gap between the sternal edges.

4. The device as set forth in claim 1, wherein the elongate member is eccentrically positioned at the most superficial level of the device, being either straight or outwardly convex, providing free space between the sternal edges.

5. The device as set forth in claim 1, wherein the elongate member join the lateral grasping ends of the device at or near its superficial aspect.

6. The device as set forth in claim 1, wherein the two outer ends of the device have substantially U shaped cross-section, shaped like two horizontally disposed horse-shoes, with their open ends towards the sternal edges, dimensioned and configured to clasp and firmly engage with the severed edges of the sternum, at a broader plane than the middle elongate member.

7. The device as set forth in claim 6 wherein the outer ends of the device are provided with strategically placed notches in its prongs and grooves along the surface of the outer ends for securing the outer ends to the sternal halves by means of suture ligation.

8. The device as set forth in claim 1, wherein the middle elongate member and the two outer ends of the device, make one continuum, without any joints, moving parts or coupling devices.

9. The device as set forth in claim 1, wherein the sternal spacing device is provided as a set of graduated, ready-to-use, sizes with small to large middle elongate member as well as the outer ends, configured to engaging with the sternal halves, of patients of various ages and body sizes.

10. The device as claimed in claims 1, 6 and 7, the prongs of the outer ends of the device can be fabricated of a Thermoplastic material like Nylon, Polycarbonates or poly-ketones so as to exert a clasping grip over the severed edges of the sternum.

11. The device as claimed in claims 1-10, wherein the device may be impregnated with suitable antibiotic coating to reduce the risk of local wound infection.

12. The device as claimed in claim 1-11, the device may be fabricated with a suitable material as to be lucent to X-rays.

13. The device as claimed in claim 1 wherein the device being provided in a set of graduated sizes to be suitable for patients of various ages and sizes.

14. The device as claimed in claim 1 wherein the device being fabricated of bio-compatible material.

15. A method for maintaining a pre-determined space between the divided edges of the Sternum or any two bony parts of the patients; the method comprising; horizontally inserting an elongate member between the two divided ends of the bony part in such a way that the two lateral end portions engage with the medial portions of the severed sternal ends and these lateral ends could further be secured by suture fixation with the sternal or divided bony ends, the fixation being facilitated by strategically placed notches and grooves in and on the lateral end portions.

16. A method for maintaining a pre-determined space between the divided edges of the Sternum or any two bony parts of the patients; the method comprising, as set forth in claim 15, by using one or multiple number of these devices which may be of different sizes in length of the middle part as well as the lateral ends, being placed at different parts of the severed sternal or bony edges.
